(19) United States
(12) Patent Application Publication
(10) Pub. No.: US 2019/0183779 A1
(43) Pub. Date: Jun. 20, 2019

(54) NAIL COATING SYSTEM

(71) Applicant: Rebecca ISA, Pepper Pike, OH (US)

(72) Inventor: Rebecca ISA, Pepper Pike, OH (US)

(21) Appl. No.: 16/318,904

(22) PCT Filed: Jul. 21, 2017
(86) PCT No.: PCT/US17/43309

§ 371 Date: Jan. 18, 2019

Related U.S. Application Data
(60) Provisional application No. 62/364,926, filed on Jul. 21, 2016.

(51) Int. Cl. A61K 8/92; A61K 8/37; A61Q 3/02

(52) U.S. Cl. CPC A61K 8/92; A61Q 3/02; A61K 8/37

(57) ABSTRACT

The present technology provides a nail coating system suitable for improving the nail plate to which it is applied. The nail coating system includes two components—an oil-based serum and a base composition. In use, the oil-based serum may be applied directly to a nail plate without first cleaning the nail plate with alcohol and/or acetone. The oil-based serum coat may fill in any cracks and crevices in the nail plate and restoring sebum and water on the nail plate. After the oil-based serum layer is buffed into the nail, the base composition may be applied for the oil-based serum coat. After the base composition dries, a user may apply a top coat, a tinted nail polish, a perfector product, etc. The resulting nail plate has improved features and restored natural sebum and water levels in the nail plate.

### CROSS-REFERENCE TO RELATED APPLICATIONS

The present application claims priority to and the benefit of U.S. Provisional Application No. 62/364,926 filed on Jul. 21, 2016, the disclosure of which is incorporated by reference herein in its entirety.

### FIELD

The present technology relates to compositions and methods for the improvement of common conditions of nails including improved adhesion, improved hydration of the nail, improved nail polish wear, improved strength of nails, rebalancing the sebum and water level of nails, improving the flexibility level in nails, improving the hardness level of nails, the prevention of nail damage, including breakage, fraying, and brittleness, along with the restoration of damaged nail plates.

### BACKGROUND

Natural nail plates, including those in the fingernails and toenails of humans, are comprised of multiple layers of translucent keratin protein. The nail plates are created within a nail matrix at the root of the nail plate and are slowly transported toward the end of the finger or toe, with the entire journey usually taking 4-6 months for fingernails and up to one year for toenails. Keratin, the fibrous structural protein of nails, is also found in human hair and skin. The keratin naturally imparts structure, toughness, durability and flexibility to the nail plates, which ensures resistance to cracking, chipping, breaking and tearing. Nail plates also contain sebum and water to maintain and protect the nail plate. The sebum and water keep the nail plate lubricated and prevent it from drying out and being fragile.

Unfortunately, nail plates can become damaged in everyday activities through exposure to harsh or corrosive chemicals, overexposure to water, mechanical damage, or due to infection or illness, such as certain autoimmune disorders. Some nail plates are also damaged through various manicure and pedicure techniques, e.g., the removal of natural oils, i.e., sebum and water with alcohol and acetone prior to adding polish to the nails. Further, the nail plates may also suffer from mechanical damage created from overly aggressive and improperly performed manicures, artificial nail or gel manicure application/removal procedures. Some people may also inherently have or develop thin/weak nail plates which easily tear, break or peel. The strength and durability of nail plates also normally decreases with advancing age. Damaged or weakened nail plates may be more susceptible to infection, excessive staining/discoloration or further continued damage. Also, as a result of advancing age, nail plates often develop grooves of varying depths which run the length of the natural nail plate. These grooves, often incorrectly perceived as ridges, can serve as weak points in the nail plate and act as seeds for initiating longitudinal splits or cracks which can develop in the plate as a result of the significant plate thinning in these grooved areas or provide for weak points in manicures for bubbling or cracking of nail polish.

Increasing or restoring the nail plate's inherent strength, flexibility, and hydration provides increased durability which allows the nail plate to better endure impacts and to resist externally applied physical forces in the form of everyday stresses, strains, etc. There is a balance that must be achieved between strength, flexibility, and durability though. An ideal nail is strong enough to resist damage but not brittle, overly flexible, etc. Further, smoothing out the grooves of a nail can allow for improved nail polish wear.

It is therefore desirable to develop a nail plate improvement system that improves strength, durability, and flexibility of nail plates while restoring a nail plate's sebum and water allowing for improved adhesion and wear of nail polish. It is also desirable to develop a nail improvement composition that it easy to apply, yet can provide long-lasting strength and durability to any type of nail plate including those which are already brittle, weak, splitting, or previously damaged. Further, it is desirable to create a nail coating system that can correct and protect the nail plates and nail plate health while also serving as a strong base for the application of cosmetic products to the nail plates.

### SUMMARY

The present technology provides a nail coating system capable of improving nail plate strength, smoothing imperfections in nail plates, restoring sebum and water in a nail plate, improving adhesion of nail lacquer for a colored manicure or pedicure, and resulting in a high quality nail treatment process without the use of chemicals that strip the nail plate such as alcohol, acetone, and/or formaldehyde after preparing a clean nail plate free of nail polish.

In one aspect, the present technology discloses a nail coating system including an oil-based serum and a base composition. The base composition may include sucrose acetate isobutyrate. Further, a coating of oil-based serum is applied directly to a nail plate coated in oil and water and the oil-based serum coating may secure the oil and water to the nail plate. The oil-based serum may also fill crevices in the nail plate. A coating of base composition may be applied to the oil-based serum coating.

In an aspect, the present technology discloses a method of using a nail coating composition including applying an oil-based serum coating to a nail plate, drying the oil-based serum coating, and applying a base composition coating to the oil-based serum coating. In an embodiment, a top coat may be applied to the base composition coating. In an embodiment, a nail polish coating may be applied to the base composition coating and followed by a top coat coating. In an embodiment, a perfector coating and top coat coating may be applied to the base composition coating.

In an aspect, the present technology discloses a method of using a nail coating composition including applying an oil-based serum coating to a nail plate with crevices, filling the crevices of the nail plate with the oil-based serum, and drying the oil-based serum coating. A base composition may be coated onto the oil-based serum coating, thereby pushing the oil-based serum into the nail plate.

These and other aspects and embodiments are further understood with reference to the following detailed description.

### BRIEF DESCRIPTION OF THE DRAWINGS

FIG. 1 is a cross-sectional view of the nail coating system on a nail surface;

FIG. 2 is a cross-sectional view of the nail coating system and a top coat on a nail surface;

FIG. 3 is a cross-sectional view of the nail coating system, nail polish, and a top coat on a nail surface;

FIG. 4 is a cross-sectional view of the nail coating system, perfector, and a top coat on a nail surface; and

FIG. 5 is a flow-chart detailing various options for using the nail coating system.

The drawings are not to scale unless otherwise noted. The drawings are for the purpose of illustrating aspects and embodiments of the present technology and are not intended to limit the technology to those aspects illustrated therein. Aspects and embodiments of the present technology can be further understood with reference to the following detailed description.

### DETAILED DESCRIPTION

The present technology provides a nail coating system capable of improving nail plate strength, smoothing imperfections in nail plates, restoring sebum and water in a nail plate, improving adhesion of nail lacquer for a colored manicure or pedicure, and resulting in a high quality nail treatment process without the use of chemicals such as alcohol, acetone, and/or formaldehyde.

### Composition

The nail coating system of the present technology includes two components—an oil-based serum component and a base component. Each component may comprise a single component or several different components.

The nail coating system includes an oil-based serum. The oil-based serum is any appropriate type of liquid or gelatinous material. The oil-based serum may be composed of any appropriate materials. The amount of materials may vary based on the batch size and the amount of other components within the oil-based serum.

The oil-based serum may be prepared by combining its component materials in any appropriate manner, e.g., sequentially, all at once, or in various stages. In an embodiment, these components may be formed in-situ. In another embodiment, the components may be preformed materials. After preparation, the oil-based serum component may be stored in any appropriate container, e.g., a standard glass nail polish container.

The nail coating system also includes a base component. The base component is a liquid component. The base component may be composed of any appropriate materials, including but not limited to, acetate, butyl acetate and/or derivatives thereof. The amount of these materials may vary based on the batch size and amount of other components within the base component. In an embodiment, the base component may be a traditional nail polish formula containing acetate, butyl acetate and/or derivatives thereof.

The base component may be prepared by combining its component materials. The oil-based serum components may be combined in any appropriate manner, e.g., sequentially, all at once, or in various stages. In an embodiment, these components may be formed in-situ. In another embodiment, the components may be preformed materials. The base component may be stored in any appropriate container, e.g., a standard glass nail polish container.

### Methods of Use

After preparation, the nail coating system may be applied to a nail via any suitable method including, but not limited to, by brush, by roller, by spraying, by dropping, etc. In an embodiment, the nail coating system may be applied by a standard brush-applicator as commonly used in nail polish application.

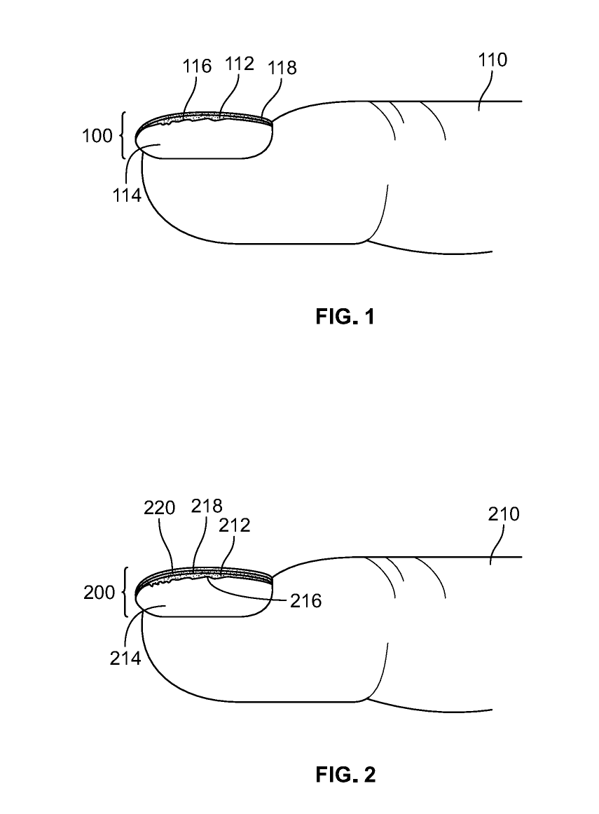

The present technology also includes a method of coating nail plates with the nail coating system 100. As shown FIG. 1, the nail coating composition may be applied to a natural nail of a finger or toe 110. The nail may be free of previous nail products, lotions, and other cosmetics products before the nail coating system is applied. Nail polish should be removed from the nail by any appropriate means, including, but not limited to acetone or other nail polish remover. The nail does not need to be further cleaned and/or dried with acetone and/or alcohol. The hands/feet, including nails, should be washed with soap and water and dried before applying the nail coating system 100.

The method of applying the nail coating system includes optionally shaking the containers holding the oil-based serum and base composition products. The target nails should be cleaned of residual nail polish by any appropriate means, including the use of acetone or other appropriate nail polish removers. Then, the hands and/or feet should be washed with soap and dried, paying attention to remove any old lotions, dirt, debris, etc. from the nails and surrounding skin. The nails may be trimmed with manicure scissors or nail clippers and cuticles and other skin may be maintained as normally done with a manicure and/or pedicure. Then, the oil-based serum 112 may be applied directly to the nail plate 114 and surrounding skin up to the knuckle, including cuticles and under the free edge of the nail. This step is counter-intuitive as in normal manicure/pedicure processes, the nail plate is first cleaned with an alcohol to remove sebum oil and cleaned with acetone to remove water from the nail plate. This step dries out the nail as removing the natural protective sebum and water leaves the nail plate dry and brittle, i.e., vulnerable to further damage.

With the present nail coating system, the oil-based serum 112 should be applied directly to the nail plate that still contains its natural sebum and water, i.e., one that has not been treated with alcohol, acetone, or any other harsh chemical and/or drying agents. Here, the oil-based serum 112 adheres directly to the nail plate 110, filling in any natural crevices 116 caused by damage from previous nail treatments, including, but not limited to, the application and removal of artificial nails or gel nail polish, or contact damage, or patterns in natural nail growth due to illness or disease. The oil-based serum 114 then creates a smooth nail plate surface, allowing for better adhesion of the next layers of nail product. The oil-based serum 114 is absorbed into the nail, locking in already existing sebum and water, and encouraging the production of additional sebum and water to help correct and restore the nail plate to its natural, healthy state.

The coat of oil-based serum 114 may be applied in any appropriate amount, enough to create a smooth covering over the user's nail plate 112. Drying time of the oil-based serum 114 may vary based on various factors, including, but not limited to, the thickness of the coat of oil-based serum, the conditions and temperature in the location of the nail coating composition process, etc. The oil-based serum 114 is then buffed with a small grit side of a buffer or any other appropriate material. The buffing process buffs down loose layers of keratin from the nail plate 112 and pushes the oil-based serum 114 into the nail plate. If all of the oil-based serum 114 is absorbed into the nail plate 112 and surrounding skin, a user may choose to apply additional coats of oil-based serum 114 after the first coat of oil-based serum 114 is absorbed. The buffing stage is repeated until the nail plate 112 stops absorbing the oil-based serum 114—a sign that an appropriate amount of oil-based serum 114 has been applied. At this point, a user will begin buffing their nail plate 112 with the smoother (e.g., leather or non-grit side of a buffer). This second buffing process further pushes the remaining oil-based serum 114 into the nail plate 112 and evenly blends the oil-based serum 114, in a process similar to buffing a shoe with stain. The time required for buffing depends on the person, the condition of their nails, the amount of keratin to be removed from the nails, and the amount of oil-based serum applied and/or remaining on the nails. Again, the stage of buffing the nails is counter-intuitive to a normal manicure/pedicure process wherein the buffing process is completed on dry nail plates. The dry buffing can damage the nail plate by removing layers of keratin, scratching the nail plate, etc. Here, the buffing of nails coated in an oil-based serum prevents the damage of nail plate and, in fact, improves the nail plate and helps to restore its natural balance of oils and water, while removing loose keratin.

After the oil-based serum coat(s) 114 has been absorbed, a coat of base composition 118 may be applied directly to the oil-based serum coat 114. The base composition 118 may further “push” the oil-based serum into the nail plate, thereby strengthening its attachment into the nail plate 112 and its crevices 116. The filling of the crevices allows for a smooth nail plate surface to apply the next layers of products. The coat of base composition 118 may be applied in any appropriate amount, enough to completely cover the oil-based serum coat on the nail. Drying time of the base composition 118 may vary based on various factors, including, but not limited to, the thickness of the coat of base composition, the conditions and temperature in the location of the nail coating composition process, etc. The base composition 118 may be dried by exposure to the environment or the finger 100 may be placed in a dryer and/or heat lamp to accelerate drying time, e.g., a UV light or an LED light. A user may choose to apply a second coat of base composition after the first coat of base composition has dried, if they do not believe it has even coated.

Once the nail coating system is applied, the coated nail plate has several unexpected resulting benefits. First, the end product is a high quality coated nail plate. The nail plate restores its natural sebum and water, therefore resulting in improved nail health such as improved strength and a less brittle, dry nail than those treated in other nail treatment systems. Second, the nail coating system is healthier to be around without the use of various chemicals that strip the nail plate, including, formaldehyde, alcohol, and acetone. Third, the coated nail plate exhibits a smooth nail plate surface. Any additional products applied to the smooth, coated nail plate will have improved adhesion and be less likely to chip and/or bubble at weakened points around bumps and crevices, resulting in a longer-lasting nail polish wear. These results are unexpected as the normal manicure/pedicure routine involves removing the natural sebum and water from a nail plate in order to create a better adhesion of additional cosmetic nail products. Further, the results are unexpected because nail polish would not be able to adhere to a nail plate having been treated with oil such as the oil-based serum of the present nail coating system. Fourth, the nail plates treated by the present nail coating system may grow out faster since they are less likely to become damaged, e.g., less cracking, splitting, peeling, etc., due to their improved strength, flexibility, hydration, and balanced sebum and water levels. This is also unique as compared to current systems which can either strength/harden nail plates or moisturize the nails. The current systems cannot do both at the same time—rather, the current systems switch back and forth between strengthening and moisturizing the nail plates—resulting in nail plates that are lacking at least some of the benefits of the present nail coating system at any given moment.

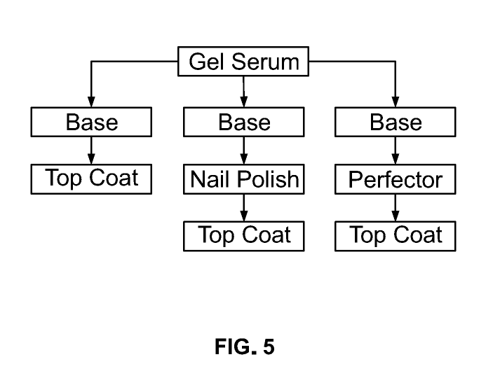

In an embodiment, shown in FIG. 2 and FIG. 5, a nail plate 214 may be treated with a nail coating system 200 through the application of oil-based serum 212, followed by buffing and the application of additional coats of oil-based serum 212, filing the nail plate crevices 216 and creating a smooth nail plate 214 surface free of loose keratin and restored to its natural balance of sebum and water. A coat of base composition 218 may be applied on top of the oil-based serum 212 coat. Lastly, a top coat 220 may be applied to the base composition coat 218. The top coat may seal in the nail coating system, and provide a shiny, manicured finish on the nail plate.

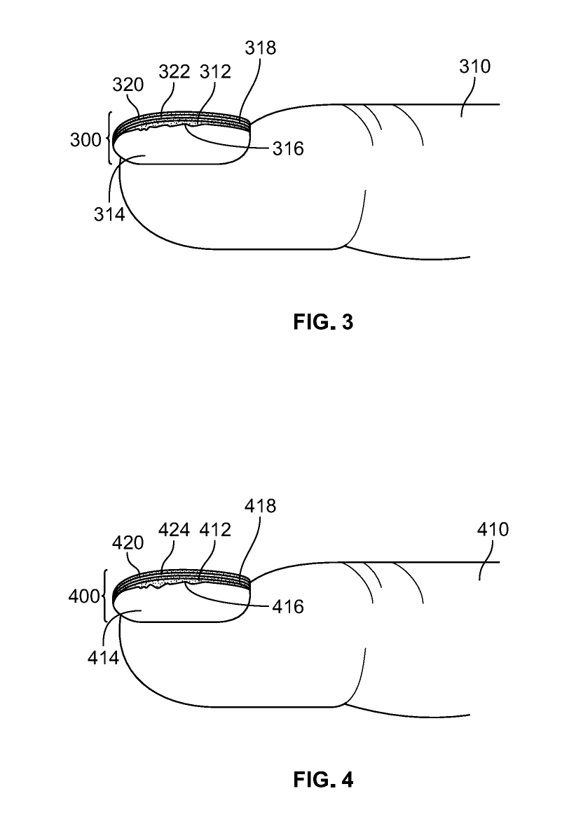

In an embodiment, shown in FIG. 3 and FIG. 5, a nail plate 314 may be treated with a nail coating system 300 through the first application of oil-based serum 312, followed by buffing and the application of additional coats of oil-based serum 312, filing the nail plate crevices 216 and creating a smooth nail plate 314 surface free of loose keratin and restored to its natural balance of sebum and water. A coat of base composition 318 may be applied on top of the oil-based serum 312 coat. A coating of standard nail polish 322, e.g., colored nail lacquer, textured nail lacquer, etc., may be applied to the coat of base composition 318. This provides a pop of color or texture to a finger or toe. Lastly, a top coat 320 may be applied to the nail polish coat 322. The top coat 320 may seal in the nail coating system and nail polish, and provide a shiny, manicured finish on the nail plate. In various embodiments, different colors, patterns, designs, etc., may be layered onto the nail plate in the nail polish coat 322.

In an embodiment, shown in FIG. 4 and FIG. 5, a nail plate 414 may be treated with a nail coating system 400 through the first application of oil-based serum 412, followed by buffing and the application of additional coats of oil-based serum 412, filing the nail plate crevices 216 and creating a smooth nail plate 414 surface free of loose keratin and restored to its natural balance of sebum and water. A coat of base composition 418 may be applied on top of the oil-based serum 412 coat. A coating of perfector 424, e.g., a composition including perfecting components, such as optical diffusers, keratin fillers, etc., may be applied to the coat of base composition 418. This provides a natural finish to a finger or toe. Lastly, a top coat 420 may be applied to the perfector coat 424. The top coat 420 may seal in the nail coating system and perfector coat, and provide a shiny, natural, manicured finish on the nail plate.

While the technology has been described with reference to various exemplary embodiments, it will be appreciated that the modifications may occur to those skilled in the art, and the present application is intended to cover such modifications and invention as fall within the spirit of the invention.

What is claimed is:

1. A nail coating system comprising:
(a) an oil-based serum; and
(b) a base composition.

2. The nail coating system of claim 1, the base composition comprises a material selected from the group consisting of: acetate, butyl acetate, or derivatives thereof.

3. The nail coating system of claim 1, wherein a coating of oil-based serum is applied directly to a clean nail plate.

4. The nail coating system of claim 3, wherein the oil-based serum coating restores sebum and water to the nail plate.

5. The nail coating system of claim 4, wherein the oil-based serum fills crevices in the nail plate.

6. The nail coating system of claim 5, wherein a coating of base composition is applied to the oil-based serum coating.

7. A method of using a nail coating composition comprising the steps of:
(a) applying an oil-based serum coating to a nail plate;
(b) buffing the oil-based serum coating into the nail plate; and
(c) applying a base composition coating to the oil-based serum coating.

8. The method of claim 7, further comprising:
applying a top coat coating to the base composition coating.

9. The method of claim 7, further comprising:
applying a nail polish coating to the base composition coating.

10. The method of claim 8, further comprising:
applying a top coat coating to the nail polish coating.

11. The method of claim 7, further comprising:
applying a perfector coating to the base composition coating.

12. The method of claim 11, further comprising:
applying a top coat coating to the perfector coating.

13. The method of claim 7, further comprising:
applying an additional coat of oil-based serum to the nail plate; and
buffing the additional coat of oil-based serum into the nail plate.

14. The method of claim 7, wherein the coated nail plate has restored sebum and water levels.

15. The method of claim 14, wherein the coated nail plate has improved nail health.

16. The method of claim 14, wherein the coated nail plate has improved strength.

17. The method of claim 9, wherein the coated nail plate has improved nail polish wear.

18. A method of using a nail coating composition comprising the steps of:
(a) applying an oil-based serum coating to a nail plate with crevices;
(b) filling the crevices of the nail plate with the oil-based serum;
(c) buffing the oil-based serum coating into the nail plate; and
(d) applying a base composition coating to the oil-based serum coating;
(e) pushing the oil-based serum into the nail plate with the base composition.

19. The method of claim 18, wherein the coated nail plate has restored sebum and water levels.

20. The method of claim 19, wherein the coated nail plate has improved nail health.
